(19) United States
(12) Patent Application Publication
(10) Pub. No.: US 2007/0298432 A1
(43) Pub. Date: Dec. 27, 2007

(54) Binding Assay Components

(75) Inventors: David Andrew Anderson, Brunswick (AU); Teresa Sylvia Howard, Malvern East (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: HEP-GENICS PTY LTD, Malvern (AU)

(21) Appl. No.: 10/578,104

(22) PCT Filed: Nov. 5, 2004
(86) PCT No.: PCT/AU04/01522

§ 371 Date: May 4, 2007

(30) Foreign Application Priority Data
Nov. 7, 2003 (AU) 2003906147

(51) Int. Cl. G01N 33/566; C12Q 1/02

(52) U.S. Cl. 435/7.1; 436/513; 436/547

(57) ABSTRACT

In one embodiment, the present invention provides a detection complex which is useful for detecting a specific analyte of interest in a sample. The complex comprises a detection marker indirectly connected to an analyte binding partner by a bridging complex. This arrangement serves to preserve or enhance the availability of analyte binding sites on the analyte binding partner and consequently enhances detection of the analyte. In some embodiments, the present invention provides a detection complex useful for detecting a specific antibody of interest in a sample. In accordance with one aspect of the present invention, methods are provided to detect one or more antibodies using a bridging complex comprising multimeric, dimeric, or chimeric molecules or particles each comprising an antigen and coupled to detection markers through the use of antibodies or a protein binding molecule, nucleic acid binding molecule, carbohydrate binding molecule or lipid binding molecule.

### BACKGROUND OF THE INVENTION

1. Field of the Invention

The present invention relates generally to the field of diagnostics. More particularly, the present invention contemplates methods for detecting an analyte such as an antibody or an antigen. The detection methods of the present invention are useful, inter alia, for diagnosis or risk determination of a medical or other condition or pre-condition, or for determination of infection status or immune status.

2. Description of the Prior Art

Bibliographic details of references in the subject specification are also listed at the end of the specification.

Reference to any prior art in this specification is not, and should not be taken as, an acknowledgement or any form of suggestion that this prior art forms part of the common general knowledge in any country.

A diverse range of assays are used in research, analysis, development and clinically to detect analytes of interest. Immunoassays are a particularly useful form of assay which exploit the specificity, strength and diversity of antibody-antigen reactions to analyse samples and detect specific components therein.

The detection of antibodies to specific antigens has been used in the diagnosis of specific disease states. For example, the presence of antibody to hepatitis A virus indicates infection with hepatitis A virus and the likelihood of immunity to subsequent infection with that virus. The detection of different classes of antibody or immunoglobulin can also provide further information about a disease or a subject's immune status. For example, a current disease state may be distinguished by the presence of IgM antibody while infection in the more distant past may be distinguished by the detection of IgG antibodies.

Methods for the detection of antibody to specific antigens are also known. For example, the enzyme-linked immunosorbent assay (ELISA) and radioimmunoassay (RIA) are routinely used in laboratories. These methods generally require some level of skill in laboratory techniques. A variety of methods have also been developed which require little skill and are rapid to perform, and which are therefore suitable for the detection of antibody to specific antigens at the point of care.

In many immunoassays, it is necessary to form a conjugate containing the specific antigen together with a detectable marker. The antigen of a virus may, for example, be conjugated with colloidal gold such that immune reactivity between the antigen-colloidal gold complex and specific antibody in a device can be detected. Alternatively, the antigen of a virus may be conjugated with an enzyme such as horseradish peroxidase, such that immune reactivity between the antigen-enzyme complex and specific antibody can be detected in an ELISA.

However, the process of conjugation between colloidal gold or enzyme and the antigen of interest may result in a reduction of the immune reactivity between the antigen and the antibody which it is intended to detect. Specifically, the antibody binding site may be the physical site of binding to the colloidal gold or enzyme such that it is inaccessible to the antibody molecule, or the process of binding may alter the conformation of the antigen such that it is no longer recognised by the antibody molecule. At the least, binding of the antigen to colloidal gold or enzyme may be in a random orientation, such that only a proportion of the antigen molecules are available to react with patient antibody to give a detectable signal in a diagnostic test.

The preparation of gold or enzyme conjugates with antigen requires the use of highly purified antigens to prevent the formation of gold or enzyme conjugates containing contaminating proteins which could then react with antibody resulting in non-specific reactions and unreliable test results. The processes used for extensive purification of antigens add to the cost of such preparations, and may also result in a reduction of immune reactivity of the antigen.

There is therefore a need for improved assay systems for detecting analytes, such as antibodies or antigens, using analyte-binding molecules with bound detection markers which do not, as a consequence of binding to the detection marker diminish the sensitivity or specificity of the assay.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In one embodiment, the present invention provides a detection complex which is useful for detecting a specific analyte of interest in a sample. The complex comprises a detection marker indirectly connected to an analyte binding partner by a bridging complex. This arrangement serves to preserve or enhance the availability of analyte binding sites on the analyte binding partner and consequently enhances detection of the analyte. In some embodiments, the present invention provides a detection complex useful for detecting a specific antibody of interest in a sample. This complex comprises a detection marker indirectly connected to an antigen component in which the antigen comprises an epitope recognised by the antibody. The detection marker is connected indirectly to the antigen by a bridging complex in order to preserve the availability of antigenic epitopes for the antibody and consequently facilitate detection of the antibody.

In accordance with one aspect of the present invention, methods are provided to detect one or more antibodies using a bridging complex comprising multimeric, dimeric, or chimeric molecules or particles each comprising an antigen and coupled to detection markers through the use of antibodies or a protein binding molecule, nucleic acid binding molecule, carbohydrate binding molecule or lipid binding molecule.

The present invention provides a set of binding partners for use in detecting an analyte which, inter alia, preserves or enhances the ability of the analyte binding partner to bind to the analyte when the analyte binding partner is connected to a detection marker. In some embodiments, the present invention provides a detection system for detecting an antibody in a sample using a detection marker-antigen complex which preserves or enhances the availability of antigenic epitopes to bind to the antibody and consequently facilitates detection thereof. The present complexes are particularly useful as part of assays, kits and other devices for screening for compounds such as specific antibodies or antigens. In an exemplary embodiment, the antigens are hepatitis viral antigens and the antibodies which bind to the hepatitis viral antigen are anti-hepatitis viral antibodies.

In some embodiments, the hepatitis viral antigens are hepatitis A virus, and/or hepatitis B virus and/or hepatitis C virus and/or hepatitis E virus.

Although the present invention is described with particular reference to detection marker-antigen complexes for use in the detection of specific antibodies, the subject invention is not so limited and extends to the use of detection marker-analyte binding partner complexes for the detection of specific analytes. The terms “antigen” and “antigenic polypeptide” include haptens and other molecules against which an antibody may be generated.

The present detection complexes may be used in combination with a large range of different immunoassays, in order to improve their sensitivity and/or specificity. In one embodiment, the analyte is immobilised on a solid support prior to exposure to the detection marker-antigen complex. In some embodiments, the complex or components of the complex may be stored in a compartment of a test kit or device. Components of the detection marker-antigen complex may be stored in separate locations or compartments.

Kits may comprise alternative detection markers, bridging partner components and analyte binding partners.

In one aspect, the detection marker-analyte binding partner arrangement and has the following structure:
M-X₂+X₁-A
wherein:
M is a detection marker indirectly linked to A to form a detection marker-analyte binding partner complex;
A is an analyte binding partner which is specifically recognised by the analyte. In some embodiments, A is an antigen bearing an epitope which is specifically recognised by an antibody to be detected. In some embodiments, A is either bound to X₂ to A is expressed as part of X₂ or occurs naturally as part of X₂;
X₁ and X₂ comprise bridge binding partners which form a bridging complex between the detection marker (M) and the analyte binding partner (A) and are bound by (+) which is a reversible non-covalent bond;
X₁ comprises a first bridge binding partner which is a particle, dimer, multimer, chimera or fusion protein comprising a portion which binds to X₂ and another portion which binds to or comprises the analyte binding partner (A) and wherein the adjacent (−) is a covalent or non-covalent bond between the first bridge binding partner and the analyte binding partner (A);

By particle is meant a viral particle or a viral like particle. In some embodiments, X₁ comprises a recombinant viral-like particle comprising a proteinaceous analyte binding partner. In some embodiments, the viral like particle is derived from an avian hepadnavirus and the antigen is expressed as a part of the L polypeptide.

X₂ comprises a second bridge binding partner which is bound, fused or otherwise directly or indirectly connected to the detectable marker (M) and wherein the adjacent (−) is a covalent or non-covalent bond. In some embodiments, X₂ is connected to the detectable marker using one or more pairs of binding molecules such as antibody-antibody biotin-strepavidin or biotin-anti-biotin antibody pairs.

In some embodiments, X₂ is an antigen binding molecule, protein binding molecule, nucleic acid binding molecule, carbohydrate binding molecule or lipid binding molecule. In another embodiment, X₂ is an antigen-binding molecule.

In another embodiment, X₂ is an antibody or an antigen-binding fragment thereof.

The analyte binding partner used in the instant arrangement may be of variable purity, as only the specific analyte binding partner in any mixture will form a complex with the detection marker. For example, a lysate of whole cells containing an antigen of interest could be used to form the complex, and only the antigen of interest would be labelled.

The detection marker-analyte binding partner complex has the advantages of a defined orientation capable of maximising the availability of binding sites for the analyte of interest. In particular, where the bridging complex comprises one or more antibodies, the antigen may be bound to the detection marker in a uniform orientation, further maximising the availability of epitopes to bind to patient antibodies.

For immunoassays, an antigen may have only a single site which is suitable for binding of patient antibody to give a result in a diagnostic test. In this situation, the binding of the detection marker to the antigen may preclude or diminish the subsequent or coincident binding of patient antibody to the same antigen species. The present invention overcomes this problem by the use of a multivalent antigen in which two or more copies of the antibody binding site are available or chimeric antigens in which the antigen of interest is physically associated with a distinct antigen or distinct epitope within the same antigen to which the colloidal gold-antibody conjugate binds. It will be evident to those skilled in the art that the detection marker may be connected to the analyte binding partner at any time up to and including the performance of the assay.

### BRIEF DESCRIPTION OF THE FIGURES

FIG. 1 is a schematic representation showing a detection marker (colloidal gold)-antigen complex comprising a dimeric ORF2.1 antigen bound via one molecule to an antibody conjugated to the detection marker leaving the second molecule of the dimer to interact with sample antibody (IgM). The IgM is immobilized on a strip containing anti-human IgM.

FIG. 2 is a schematic representation showing a detection marker (colloidal gold) antigen complex comprising a hepatitis A virus (HAV) particle (first bridging binding partner) which by its multimeric nature also comprises the antigen bearing an epitope recognised by immobilized IgM antibody and a monoclonal anti-HAV antibody (second bridge binding partner) conjugated to the detection marker. Under defined conditions such as virus concentration and time of incubation, only one or a few copies of the epitope within each virus will react with the monoclonal antibody bound to the colloidal gold, leaving the remaining epitopes on the virus particle to react with patient antibody. The IgM is immobilized on a strip containing anti-human IgM.

FIG. 3 is a schematic representation showing a detection marker (colloidal gold) antigen complex comprising a virus-like particle (VLP) of duck hepatitis B virus (DHBV) (the first bridging binding partner) comprising an antigen bearing an epitope recognised by immobilized IgM patient antibody and a monoclonal anti-DHBV S antigen antibody (second bridge binding partner) which recognises a second epitope on the VLP S antigen, conjugated to the detection marker. The monoclonal antibody conjugated to colloidal gold (McAb 7C12) is directed to an epitope in the DHBV part of the VLP (the S antigen) rather than the analyte binding partner antigen, leaving copies of the antigen on the VLP to react with patient antibody to give a visible signal in a diagnostic test. The IgM is immobilized on a strip containing anti-human IgM.

FIG. 4 is a schematic representation showing a detection marker (colloidal gold) antigen complex comprising a virus-like particle (VLP) of duck hepatitis B virus (DHBV) (the first bridging binding partner) comprising an antigen bearing an epitope recognised by immobilized IgM patient antibody and a monoclonal antibody (second bridge binding partner) which recognises the same epitope on the analyte binding antigen conjugated to the detection marker. The monoclonal antibody conjugated to colloidal gold is directed to the analyte binding partner antigen, but due to the three-dimensional structure of the VLP with copies of the epitope spread over its surface, only one or a few copies of the epitope within each VLP will react with the monoclonal antibody leaving the remaining copies within the VLP to bind to patient antibody to give a visible signal in a diagnostic test. The IgM is immobilized on a strip containing anti-human IgM.

FIG. 5 is a schematic representation showing a detection marker (colloidal gold)-antigen complex comprising a monomeric antigen bound via one epitope to an antibody conjugated to the detection marker leaving a second epitope of the monomer to interact with sample antibody (IgM). The IgM is immobilized on a strip containing anti-human IgM.

FIG. 6 is a schematic representation showing a detection marker (colloidal gold)-antigen complex comprising a chimeric recombinant fusion protein comprising mannose binding protein fused to the analyte binding antigen (first bridge binding partner) and a monoclonal antibody to mannose binding protein (second bridge binding partner) conjugated to colloidal gold. As the monoclonal antibody is directed to MBP, the entire analyte antigen is free to react with sample antibody. The sample antibody IgM is immobilized on a strip containing anti-human IgM.

FIG. 7 is a schematic representation showing a detection marker (colloidal gold)-antigen complex comprising a chimeric recombinant fusion protein comprising mannose binding protein fused to the analyte binding antigen (first bridge binding partner) and a ligand (mannose) to mannose binding protein (MBP) (second bridge binding partner) conjugated to colloidal gold. As the ligand is directed to MBP, the entire analyte antigen is free to react with sample antibody. The sample antibody IgM is immobilized on a strip containing anti-human IgM.

FIG. 8 is a schematic representation showing detection of IgM antibodies to hepatitis A. Specifically, a detection marker (colloidal gold) is connected to an analyte binding protein (HAV particles) by bridge binding partners and using protein:protein binding molecules (biotin:anti-biotin antibody) to connect the detection marker with the second bridge binding partner (X₂). Anti-HAV monoclonal antibody is used to bind to hepatitis A virus particles (an example of an X₁ comprising a particle or a multimer) which are capable of binding to the antibody of interest (IgM antibodies to hepatitis A). Colloidial gold is conjugated to anti-biotin antibodies which recognise biotinylated anti-HAV monoclonal antibodies. In use, only a few copies of the epitope within each viral particle will react with the anti-HAV antibodies.

### DETAILED DESCRIPTION OF THE PREFERRED EMBODIMENTS

The present invention provides a system for use in the detection of an analyte in assays and more particularly the detection of antibodies in immunoassays.

The present invention provides methods for detecting an analyte in a sample, the method comprising contacting the sample with a detection marker-analyte binding partner complex in which said detection marker is connected indirectly to the analyte binding partner by a bridging complex to preserve or enhance the availability of binding sites for the analyte; and detecting the detection marker to indicate the presence of the analyte in the sample.

As used herein, the singular forms “a”, “an” and “the” include plural aspects unless the context clearly dictates otherwise. Thus, for example, reference to an “antibody” includes a single antibody or antibody species, as well as two or more antibodies of the same or different specificity; reference to a “sample” includes two or multiple samples; and so forth.

The detection marker-analyte complexes of the present invention are detected using art recognized methods.

As used herein, reference to “detecting” is meant in its broadest sense to include assays which qualitatively or quantitatively test for the presence of analyte. Persons of skill in the art will recognise that there are a large range of assays which are suitable for use with the complexes of the present invention. Chromatographic assays are particularly sophisticated and a large number of different formats are available which are tailored to the prevailing reagents and instruments and the outcomes required in any particular investigation. “Rapid” assays, using chromatographic principles, are tailored for accuracy, speed and ease of use. The complexes of the present invention are particularly suited to use immunochromatographic devices. With appropriate detection markers however, the present complexes are also suitable for analysis in a range of different formats. Immunoassay or enzyme-based chromatographic assays are particularly preferred and these are described in Wild D “The Immunoassay Handbook”, Nature Publishing Group, 2001 and by reference to U.S. Pat. Nos. 4,016,043; 4,590,159; 5,714,389; 5,877,028, 5,922,537, 6,168,956 and 6,548,309 incorporated herein and information disclosed by references cited therein. For immunochromatographic assays, an analyte of interest is detected by agglutination with an antibody to the analyte which antibody is also linked to a detection marker. Analogous enzyme-based assays use an enzyme reaction in place of an antigen-antibody interaction. Various modifications of immunochromatographic methods are described in Published US Patent Application Nos. 20010006821, 20040087036 and 20040214347 which are incorporated herein in their entirety. Immunogold filtration methods for multiple analyte analyses are described in Published US Patent Application No. 20030165970 incorporated herein.

A wide range of “detection markers” have been described and are suitable for use in the present invention. Detection may be on the basis of any analytically identifiable physical or chemical property of the marker which allows detection of the complex. Thus the marker may be a mass tag, it may be radioactive, or identifiable by colour, spectroscopy or its magnetic or paramagnetic properties. In many assays the detection of analyte involves spatial separation of bound and unbound detection complexes. Alternatively, the detection marker may produce a distinguishable signal only when connected to the analyte of interest. Colloidal conjugates are particularly preferred.

Convenient detection markers for the instant assays include without limitation: chemiluminophores such as acridinium ester, acridinium sulphonamide, isoluminol; coenzymes such as ATP, FAD, NAD; electrochemiluminophores such as ruthenium tris(bipyridul); enzymes such as acetate kinase, alkaline phosphatase, β-lactamase, glucose oxidase, firefly luciferase, β-D-galactosidase, horseradish peroxide, glucose 6-phosphate dehydrogenase, laccase, Renilla luciferase, xanthine oxidase; fluorophores such as europium trisbipyridine cryptate (and other lanthanide cryptates), fluorescein, β-phycoerythrin, rhodamine, umbelliferone derivatives, Texas Red; free radicals such as nitroxide; fusion conjugates such as alkaline phosphatase—anti-phytochrome single chain antibody; alkaline phosphatase—basic fibroblast growth factor receptor; apoaequorin—IgG heavy chain; bacterial alkaline phosphatase—IgG Fc binding protein; firefly luciferase—protein A; human placental alkaline phosphatase—4-1 BB ligand; marine bacterial luciferase (β-subunit)—protein A; metapyrocatechase—protein A; protein A—antiphytochrome single chain antibody; Pyrophorus plagiophthalamus luciferase—protein A; genes such as firefly luciferase; metal and metalloid such as gold, silver, selenium; metal complexes such as cyclopentadienylmanganese(I) tricarbonyl, gold cluster; microparticles such as latex, erythrocytes, liposomes; nucleic acids such as pUC19 DNA; phosphors such as europium-activated yttrium oxisulfide; photoproteins such as aequorin; quantum dots such as zinc sulfide-coated CdSe microparticle; radioisotopes such as ¹²⁵I; redox complexes such as ferrocene; substrates such as galactosyl umbelliferone and virus such as bacteriophage T4.

Although not wishing to limit the invention to any particular detection marker and mode of detection, the use of flow cytometry is particularly convenient in high throughput systems. As is known in the art, flow cytometry is a high throughput technique which involves rapidly analyzing the physical and chemical characteristics of particles as they pass through the path of one or more laser beams while suspended in a fluid stream. As each cell or particle intercepts the laser beam, the scattered light and fluorescent light emitted by each cell or particle is detected and recorded using any suitable tracking algorithm. The use of fluorophores is particularly useful. Examples of suitable fluorophores may be selected from the list given in Table 1. Other detectable markers for use in this format include luminescence and phosphorescence as well as infrared dyes as mentioned above.

TABLE 1

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3,5 | 581 | 596; (640) |
| Cy5 | (625); 650 | 670 |
| Cy5,5 | 675 | 694 |
| Cy7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bormide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Cell function probes

| | | |
|---|---|---|
| Indo-1 | 361/330 | 490/405 |
| Fluo-3 | 506 | 526 |
| DCFH | 505 | 535 |
| DHR | 505 | 534 |
| SNARF | 548/579 | 587/635 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²Em: Peak emission wavelength (nm)

Any suitable method of analyzing fluorescence emission is encompassed by the present invention. In this regard, the invention contemplates techniques including but not restricted to 2-photon and 3-photon time resolved fluorescence spectroscopy as, for example, disclosed by Lakowicz et al. Biophys. J. 72: 567, 1997, incorporated herein by reference), fluorescence lifetime imaging as, for example, disclosed by Eriksson et al. (Biophys. J. 2: 64, 1993, incorporated herein by reference) and fluorescence resonance energy transfer as, for example, disclosed by Youvan et al. (Biotechnology et elia 3: 1-18, 1997).

An “analyte” includes any molecule of biological interest and includes without limitation: cytokines, hormones, antigens, forensic samples, antibodies, haptens, enzymes, natural products, components of chemical libraries, drugs including those of veterinary or pharmaceutical interest, environmental constituents and the like.

Antigens are generally required in purified form and are often conveniently produced recombinantly. However, the antigen of the present invention may be naturally occurring synthetic, recombinant, carbohydrate, lipid, or drug molecules. The size and composition of the expressed molecule is usually determined by reference to the antibodies with which it is required to react. If the antigen is too complex, it is likely to comprise binding sites for antibodies which are not required to be detected. Accordingly the term antigen is used herein as a reference to the epitope bearing portion of a molecule when in proteinaceous form. The term does not exclude modification to a polypeptide or proteinaceous molecule and including myristilation, glycosylation, phosphorylation and the like. Included within the definition are, for example, polypeptides containing one or more analogs of an amino acid (including for example, unnatural amino acids such as those given in Table 2) or polypeptides with substituted linkages. Reference to a polypeptide or protein means a polymer of amino acids and should not be limited to any particular length. The term, therefore, includes an epitope, peptide, polypeptide, protein or proteinaceous molecule of any length. The antigenic polypeptide may comprise single epitope regions through to multiple epitope regions including repeated epitope regions. The antigenic polypeptide may derive from a single or multiple sources although antigens from infectious agents, such as, for example, viruses, bacteria, fungi, protozoa, trematodes, nematodes, prions and the like are contemplated, as are tumour-related antigens. Antigenic regions of many agents and tumour-related proteins are well known in the art.

TABLE 2
Codes for non-conventional amino acids

| Non-conventional amino acid | Code |
|---|---|
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids and the introduction of double bonds between C_{α} and C_{β} atoms of amino acids.

The terms “fusion polypeptide” or “chimeric polypeptide” or “hybrid polypeptide” are interchangeably used to mean a polypeptide comprising two or more associated polypeptides which are expressed as part of the same expression product, or which are generated by synthetic means. Fusion polypeptides may comprise two or more polypeptides and intervening regions such as, for example, linker or spacer regions. In particular, regions which permit or directly or indirectly facilitate a particular surface topology may be selected. Polypeptide topology in a viral particle may be assessed for example by protease protection assay or by determining interactivity with antibodies. Accordingly, the term “fusion” in “fusion polypeptide” is not used in the sense of “viral fusion”.

“Subject” as used herein refers to an animal, preferably a mammal and more preferably human. A patient regardless of whether a human or non-human animal may be referred to as an individual, subject, animal, host or recipient. The molecules and methods of the present invention have applications in human medicine, veterinary medicine as well as in general, domestic or wild animal husbandry. For convenience, an “animal” includes an avian species such as a poultry bird, an aviary bird or game bird. The preferred animals are humans or other primates, livestock animals, laboratory test animals, companion animals or captive wild animals.

Examples of laboratory test animals include ducks, snow geese, mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species, zebrafish and amphibians are also contemplated.

The antigen may comprise epitope regions from two or more polypeptides from different organisms, species or subspecies.

The term “sample” is used in its broadest context to include purified or unpurified compositions from a subject, laboratory or environment. In a preferred embodiment, the sample is a biological sample collected from an antibody containing fluid from a subject and may include without limitation tissue or cells from any tissue such as blood, plasma, lymph, saliva or other mucous secretions, tears, spinal fluid and so forth. It should be understood that reference to a sample includes samples which have undergone some form of processing as well as samples taken directly from a subject, environment or laboratory. Processing may include such steps as dilution, filtration or other separation techniques or maceration.

The terms “binding” “conjugation” “complex” “connection” “bond” are used interchangeably herein unless otherwise stated. The component parts of the instant complex may be linked by a range of different chemical bonds. In some embodiments, one important limitation is that the complex remains intact for the purpose of the assay. A covalent bond between component parts is essentially a non-reversible bond. Antibody-antigen and ligand-ligand binding partner bonds are generally non-covalent however, the components are selected on the basis that they survive the required The terms “antigen” and “antigenic polypeptide” include haptens and other molecules against which an antibody may be generated. assay and storage conditions.

Reference to preserving or enhancing the availability of binding sites means relative to the availability of binding sites if the analyte binding partner were conjugated to the detection marker either directly or via an antibody. Specifically, by using multimeric, dimeric, chimeric, fusion or viral particle molecules linked in accordance to the present invention to the analyte binding molecule, binding sites of the analyte binding molecule are reserved for binding to the analyte.

Fusion proteins comprising an analyte binding molecule such as an antigen may generally be produced using well known techniques, such as those summarised in molecular biology laboratory manuals for example, Sambrook and Russell “Molecular Cloning—A Laboratory Manual” (Cold Spring Harbour Press, 2001 incorporated herein by reference). Fusion proteins consist of a sequence of amino acids of interest covalently attached at their amino or carboxy termini to one or more carrier sequences. Either the carrier sequence or the sequence of amino acids may comprise an analyte binding protein. If the carrier sequence carries the antigenic epitopes, epitope tagging method may conveniently be used. Expression systems and vectors are also described in Sambrook and Russell (supra) together with purification and re-folding protocols. Specifically, expression systems may use for example, bacterial, mammalian, yeast or insect host cells depending on the size and nature of the analyte binding molecule to be expressed. A wide range of plasmids are commercially available for the expression of fusion proteins. Chimeric proteins and multimeric molecules comprising an analyte binding molecule fused to the first bridge binding partner polypeptide are generated using equivalent procedures.

Reference to “particle” herein is a reference to a viral particle or a viral-like particle.

Viral particles and viral-like particles (VLPs) are produced by standard procedures known in the art. VLPs mimic the capsids or envelopes of native virions and may be obtained by recombinant expression of capsid or envelope proteins in, for example vaccinia (Hagensee et al., J. Virol. 67: 315, 1993) or in baculoviruses (Rose et al., J. Virol. 67: 1936, 1993). The hepatitis B virus (HBV) subviral particle (HBsAg-S) has been viewed as a candidate to produce recombinant VLPs. Several studies have examined which domains are suitable for insertion of foreign epitopes (Bruss et al., J. Virol. 65:3813-3820, 1994; Delpeyroux et al J. Mol. Biol. 195:343-350, 1987), including N terminal fusion of the preS domain (Prange, et al, J. Gen. Virol. 76:2131-2140, 1995).

Antigens may generally be identified using well known techniques, such as those summarized in Paul, “Fundamental Immunology”, 3rd edition., 243-247 (Raven Press, 1993) and references cited therein. Such techniques include screening polypeptides and overlapping fragments for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Antigen fragments may react at a level that is similar to or greater than the reactivity of the full length polypeptide. Screens may generally be performed using methods well known to those of ordinary skill in the art, such as those described in Harlow and Lane, “Antibodies: A Laboratory Manual” (Cold Spring Harbor Laboratory, 1988). For example, a polypeptide may be immobilized on a solid support and contacted with patient sera to allow binding of antibodies within the sera to the immobilized polypeptide. Unbound sera may then be removed and bound antibodies detected, for example using a labeled Protein A.

The term “binding partner” or “binding pair” is a reference to complementary molecules which bind or interact with each other via a reversible non-covalent or covalent attachment determined by their structure. Exemplary proteinaceous binding partners include antibody-antigen, enzyme-substrate, biotin-streptavidin, mannose/maltose/amylose-mannose/maltose/amylose-binding protein and cytokine or ligand receptor interactions.

Monoclonal antibodies are conveniently prepared in pure form and in large quantities. The preparation of hybridoma cell lines for monoclonal antibody production derived by fusing sensitized lymphocytes with an immortal cell line and selecting specific antibody producers is well known in the art by now standard procedures such as those described in Harlow and Lane (supra); and Kohler and Milstein, European Journal of Immunology 6: 511-519, 1976.

In another aspect, the present invention provides a method for detecting an antibody in a sample comprising contacting said antibody with a detection marker-antigen complex wherein the antigen comprises an epitope which is specifically recognised by the antibody, and wherein said detection marker is connected indirectly to said antigen in order to preserve the availability of epitopes on said antigen; and detecting said analyte.

In relation to this embodiment, the detection marker is connected indirectly to said analyte binding partner by a bridging complex comprising a binding pair wherein the first partner of the bridge binding pair is a particle, complex, multimer or fusion protein comprising said analyte binding partner and the second bridge binding partner is conjugated or fused or otherwise connected to said detectable marker.

Accordingly, the present invention further provides a method for detecting an antibody in a sample comprising contacting said antibody with a detection marker-antigen complex which antigen comprises an epitope which is specifically recognised by said antibody, wherein said detection marker is connected indirectly to said antigen in order to preserve the availability of epitopes on said antigen and wherein the indirect connection is by a bridging complex comprising a binding pair wherein the first partner of the bridge binding pair is a particle, complex, multimer including dimer, chimera or fusion protein or equivalent structure comprising said antigen and the second partner of the bridge binding pair is conjugated or otherwise fused to said detectable marker; and detecting the analyte.

In a related aspect, the detection marker-analyte binding partner complex comprises the following structure:
M-X₂+X₁-A
wherein:
M is a detection marker indirectly linked to A to form the detection marker-analyte binding partner complex;
A is a analyte binding partner which is specifically recognised by the analyte. In one embodiment, A is an antigen bearing an epitope which is specifically recognised by an antibody present in a patient sample;
X₁ and X₂ are bridge binding partners which form the bridging complex between the detection marker (M) and the analyte binding partner (A) and are bound by (+) which is a reversible non-covalent bond;
X₁ comprises a first bridge binding partner which is a particle, complex, dimer, multimer or fusion protein comprising a portion which binds to X₂ and another portion which binds to the analyte binding partner (A) and the adjacent (−) is a covalent or non-covalent bond between the first bridge binding partner and the analyte binding partner (A);
X₂ comprises a second bridge binding partner which is bound, fused or otherwise connected to the detection marker (M) and the adjacent (−) is a covalent or non-covalent bond.

In some embodiments, X₂ is connected to the detectable marker using one or more pairs of binding molecules such as biotin-strepavidin or biotin-anti-biotin antibody.

In some embodiments, X₂ is an antigen binding molecule, protein binding molecule, nucleic acid binding molecule, carbohydrate binding molecule or lipid binding molecule. In another embodiment, X₂ is an antigen-binding molecule.

In another embodiment, X₂ is an antibody or an antigen-binding fragment thereof.

In some embodiments, the antibody or antigen binding fragment thereof may advantageously have the same or a different specificity as the antibody to be analysed.

In some embodiments, the second binding partner conjugated or otherwise attached to the detectable marker is a monoclonal antibody which recognises the same immunodominant epitope recognised by the specific sample antibody to be analysed.

In a preferred aspect, the first bridge partner, X₂ comprises a dimeric or multimeric form of the antigen.

In a further aspect, the first bridge binding partner (X₂) is a viral particle or virus-like particle. Preferred virus particles are derived from hepadnaviruses. Preferred virus-like particles are derived from duck hepatitis B virus as shown, for example, diagrammatically in the Examples.

In a further embodiment, the second bridge binding partner comprises a carbohydrate and the fusion protein comprising the antigen also comprises a carbohydrate binding protein. In some embodiments, the second bridge binding partner comprises mannose and the fusion protein comprising the antigen comprises a mannose binding protein.

In another aspect the present invention provides a kit for detecting a specific antibody in a sample, in compartmental form comprising a portion to receive the sample, and a portion to receive a detection marker-antigen complex wherein the antigen comprises an epitope which is capable of being recognised by said specific antibody, if present in the sample, and wherein said detection marker is connected indirectly to said antigen in order to preserve the availability of antigenic epitopes to said antibody and detection thereof relative to a control.

In a preferred embodiment, of this aspect of the invention the detection marker is connected indirectly to the antigen by a bridging complex comprising a binding pair wherein the first partner of the bridge binding pair is a particle, complex, dimer, multimer or fusion protein comprising said antigen and the second partner of the bridge binding pair is conjugated or otherwise connected to said detectable marker.

Accordingly to this aspect, the detection marker-analyte binding partner complex comprises a bridging complex and has the following structure:
M-X₂+X₁-A
wherein:
M is a detection marker indirectly linked to A to form the detection marker-analyte binding partner complex;
A is an analyte binding partner which is specifically recognised by the analyte. In one embodiment, A is an antigen bearing an epitope which is specifically recognised by an antibody present in a patient sample;
X₁ and X₂ comprise bridge binding partners which form the bridging complex between the detection marker (M) and the binding partner (A) and are bound by (+) which is a reversible non-covalent bond;
X₁ comprises a first bridge binding partner which is a particle, complex, dimer, multimer or fusion protein comprising a portion which binds to X₂ and another portion which binds to the analyte binding partner (A) and wherein the adjacent (−) is a covalent or non-covalent bond between the first bridge binding partner and the analyte binding partner (A);
X₂ comprises a second bridge binding partner which is also bound, fused or otherwise connected to the detectable marker (M) and wherein the adjacent (−) is a covalent or non-covalent bond.

In some embodiments, X₂ comprises a protein binding molecule, carbohydrate binding molecule, nucleic acid binding molecule, or lipid binding molecule.

In another embodiment, X₂ comprises an antibody or an antigen-binding molecule.

In one embodiment, the detection marker-analyte binding partner complex and has the following structure:
M₁-X₂+X₁-A₁
wherein:
M₁ is a visibly, optically or magnetically or other instrumentally or chemically detectable marker indirectly linked to A to form the detection marker-antigen complex;
A₁ is an antigen bearing an epitope which is specifically recognised by an antibody present in a patient sample;
X₁ and X₂ comprise bridge binding partners which form the bridging complex between the detection marker (M₁) and the binding partner (A₁) and are bound by (+) which is a reversible non-covalent bond;
X₁ comprises a first bridge binding partner which is a particle, complex, dimer, multimer or fusion protein comprising a portion which binds to X₂ and another portion which binds to or comprises the antigen (A₁) and wherein the adjacent (−) is a covalent or non-covalent bond between the first bridge binding partner and the antigen (A₁);
X₂ comprises a second bridge binding partner which is a antibody or antigen-binding molecule conjugated or otherwise connected to a visibly, optically or magnetically or other instrumentally or chemically detectable marker (M₁) and wherein the adjacent (−) is a covalent or non-covalent bond.

In some embodiments, the kit is a chromatographic including an immunochromatographic kit and the analyte is immobilized to a solid support to facilitate its detection.

The kit may alternatively or in addition comprise separate compartments holding the detection marker-second bridge binding partner complex and said first bridge binding partner-antigen complex. In some embodiments, one or each of the detection markers, X₂, X₁ and the analyte binding partner are stored in separate compartments. If these components are stored separately in the kit, they may be combined before or during the assay procedure. The components may be stored in solution, in dried, frozen or freeze-dried form.

In a particularly preferred embodiment, colloidal gold-monoclonal antibody conjugate may be mixed with the cognate antigen prior to addition to the device during manufacture. In the example of hepatitis E virus, colloidal gold conjugated with monoclonal antibody 4B2 (Riddell, M. A., et al J. Virol. 74:8011-8017, 2000) is mixed with an equivalent volume of recombinant HEV antigen ORF2.1, and allowed to incubate at about 15-37° C. before addition to the “conjugate pad” of the device. The reagents are then dried, and following rehydration the pre-formed complex is available to react with immobilised anti-HEV specific IgM in the device.

Alternatively, the colloidal gold-monoclonal antibody conjugate and antigen may be physically separated during manufacture of the device, and allowed to mix and form complexes during performance of the assay. In the example of hepatitis A virus, colloidal gold conjugated with monoclonal antibody K3-4C8 (MacGregor A. et al, J. Clin. Microbiol., 18(5):1237-1243, 1983) is added to the “conjugate pad” of the device, while the inactivated whole virus HAV antigen is added separately to the “virus pad” and the reagents are then dried. During performance of the assay, the “conjugate pad” is first rehydrated and then comes into contact with the “virus pad” during performance of the assay, allowing rehydration of the virus. Complexes are newly formed during this process and are then available to react with immobilised anti-HAV specific IgM in the device. Alternatively, the colloidal gold-monoclonal antibody conjugate may be prepared by indirect methods such as without limitation the use of colloidal gold-antibiotin antibody conjugate and biotin-monoclonal antibody conjugate which, when mixed, will form a non-covalent complex of colloidal gold-monoclonal antibody conjugate.

The components of the kit are conveniently stored in dry and/or frozen form and are reconstituted prior to use.

In a preferred embodiment, the specific antibody in a patient sample is immobilized. Antibody may be conveniently immobilized to a solid support using an anti-species antibody that may also be specific for particular antibody isotypes such as IgM, IgA, IgE or IgG. A large number of different solid supports are now well known in the art and include beads, particles, plates, membranes, filters, tubes etc.

The detection complexes of the present invention are particularly suited either indvidually or together as components in high throughput or multiplexed assays capable of analysing multiple samples using multiple detection complexes. Preferably such assays are automated and/or controlled by computer software.

The present invention is further described by the following non-limiting Examples.

### EXAMPLE 1

### Dimeric ORF2.1 Antigen of Hepatitis E Virus

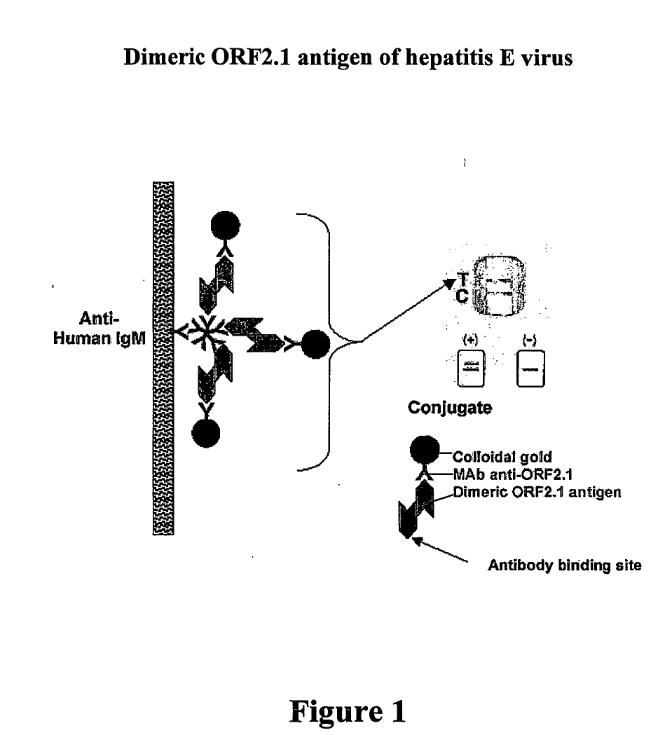

In this example the colloidal gold-antibody conjugate is complexed with dimeric hepatitis E virus ORF2.1 antigen before the conjugate is applied to the device during manufacture. The monoclonal antibody (McAb 4B2) may be directed against the immunodominant epitope in the antigen of interest, and in the presence of saturating amounts of antigen only one molecule within the dimer will react with monoclonal antibody bound to the colloidal gold, leaving the second molecule within the dimer to react with patient antibody to give a visible signal in a diagnostic test as represented schematically in FIG. 1. In the examples, the patient antibody is IgM to indicate current or recent infection with the disease organism encoding the antigen of interest, but it is evident that the methods could be equally used for other classes of antibody (such as IgG or IgA or IgE) by substitution of the appropriate anti-immunoglobulin antibody on the solid phase which may comprise flat, planar, round or curved surfaces. The ORF2.1 recombinant antigen is described in Li, F et al. J Med. Virol. 52:289-300, 1997; Anderson D. A. et al., J. Virol. Methods. 81:131-142, 1999; Li, F. et al., J Med. Virol. 60:379-386, 2000; and Riddell, M. A., et al (supra).

### EXAMPLE 2

### Multimeric Antigen of Hepatitis A Virus

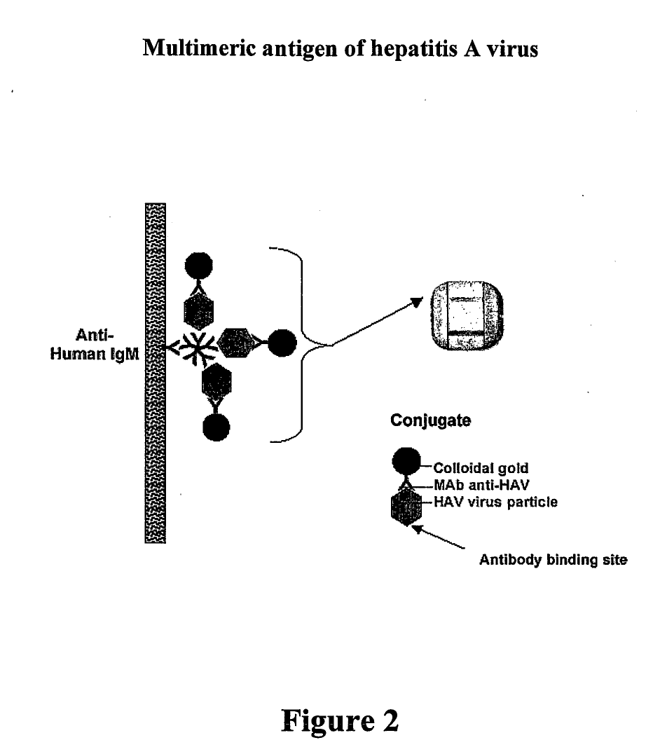

The colloidal gold-antibody conjugate is complexed with hepatitis A virus particles (antigen) during performance of the assay, by bringing together the separate assay compartments containing the two parts. In this example, the monoclonal antibody (K34C8) may also be directed against the immunodominant epitope in the antigen of interest (virus), but under defined conditions such as virus concentration and time of incubation only one or a few copies of the epitope within each virus particle will react with monoclonal antibody bound to the colloidal gold, leaving the remaining epitopes within the virus particle to react with patient antibody to give a visible signal in a diagnostic test as shown schematically in FIG. 2.

### EXAMPLE 3

### Virus-Like Particle (VLP) of Duck Hepatitis Virus

### A. Use of Anti-DHBV Bridge

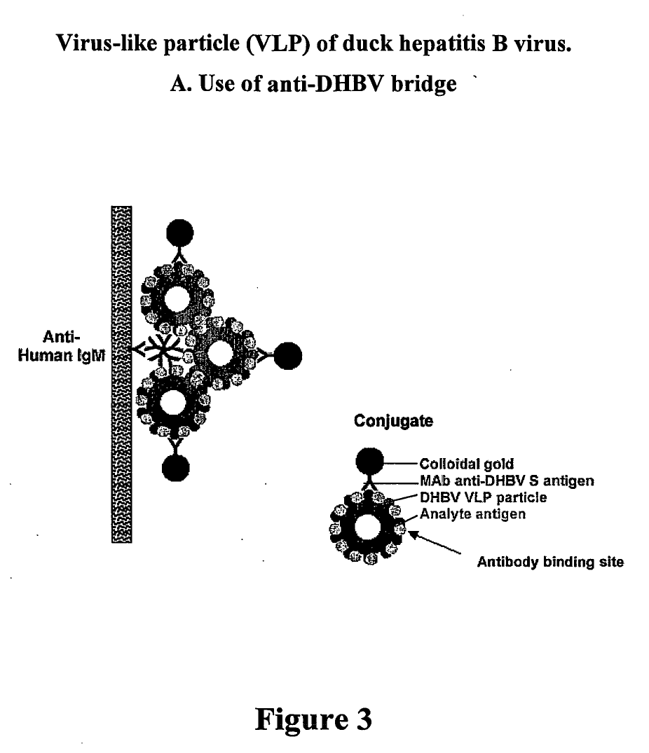

In this example, the colloidal gold-antibody conjugate may be preferentially complexed with virus-like particles (VLPs) of duck hepatitis B virus (DHBV) in which the antigen of interest is expressed as part of the chimeric VLP (described in International Publication No. WO 2004/092387 in the name of Hepgenics Pty Ltd). In this example, the monoclonal antibody which is conjugated to colloidal gold (7C12) is directed against an epitope in the DHBV part of the VLP (the S or L antigen) rather than in the antigen of interest, thereby leaving copies of the antigen of interest within the VLP to react with patient antibody to give a visible signal in a diagnostic test as shown schematically in FIG. 3.

### EXAMPLE 4

### Virus-Like Particle (VLP) of Duck Hepatitis Virus

### B. Use of Anti-Analyte Bridge

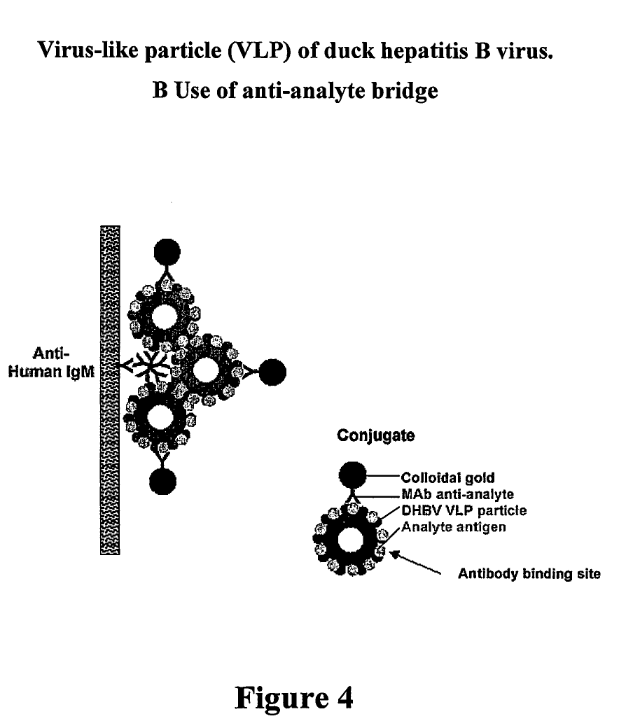

In this example the colloidal gold-antibody conjugate may again be preferentially complexed with virus-like particles (VLPs) of duck hepatitis B virus in which the antigen of interest is expressed as part of the chimeric VLP (described in International Publication No. WO 2004/092387 in the name of Hepgenics Pty Ltd). In this example, the monoclonal antibody which is conjugated to colloidal gold may be directed against the immunodominant epitope in the antigen of interest, but due to the 3-dimensional structure of the VLP with copies of the epitope spread over its surface, only one or a few copies of the epitope within each VLP will react with monoclonal antibody bound to the colloidal gold, leaving the remaining epitopes within the VLP to react with patient antibody to give a visible signal in a diagnostic test as represented schematically in FIG. 4.

### EXAMPLE 5

### Monomeric Antigen with Second Binding Site as Bridge

### A. Use of Second Epitope on the Analyte Antigen

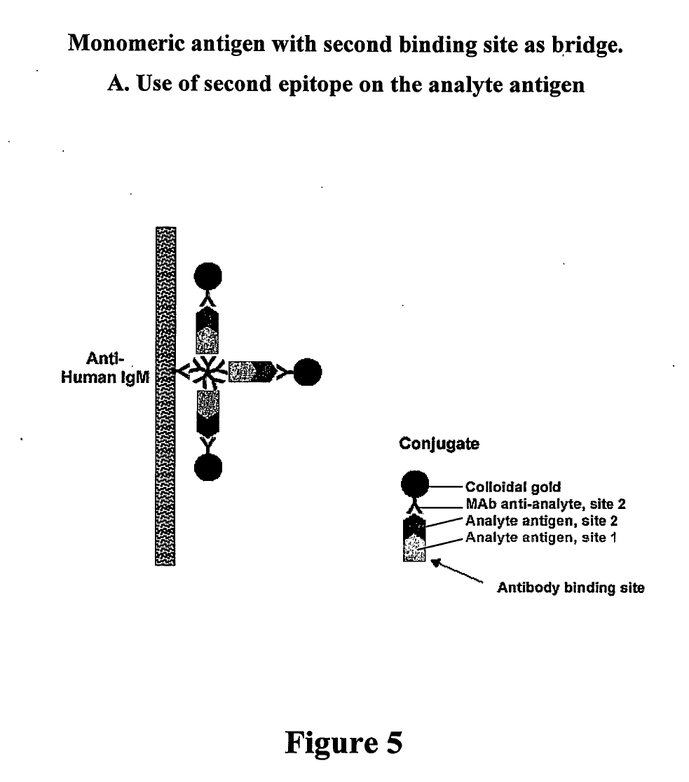

In this fifth example, the colloidal gold-antibody conjugate may be complexed with a monomeric antigen. In this example, the monoclonal antibody which is conjugated to colloidal gold is not directed against the immunodominant epitope in the antigen of interest, but instead is directed against a separate epitope in the antigen of interest, leaving the immunodominant epitope(s) to react with patient antibody to give a visible signal in a diagnostic test as shown schematically in FIG. 5.

### EXAMPLE 6

### Monomeric Antigen with Second Binding Site as Bridge

### B. Use of Fusion Protein Such as Mannose Binding Protein (MBP) with Analyte Bridge

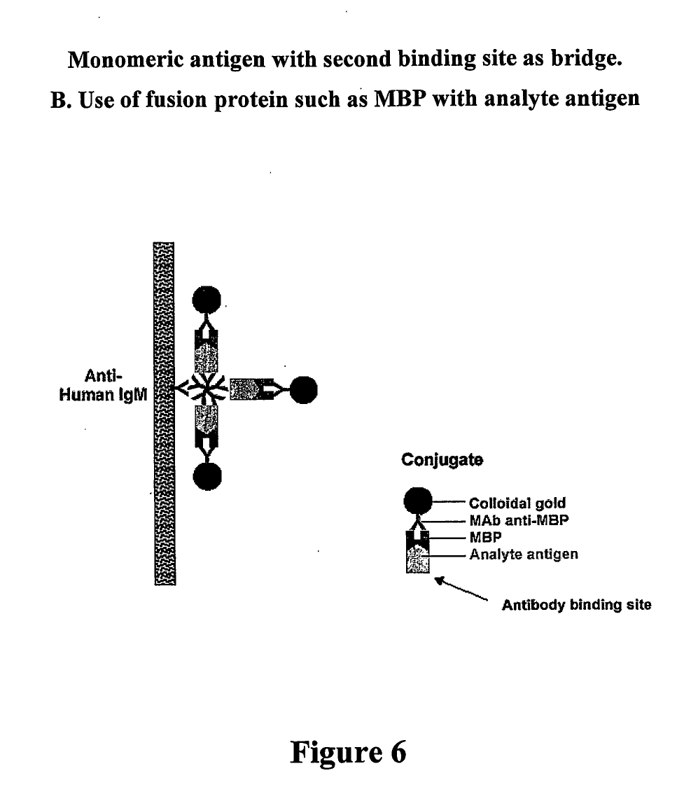

This example also applies to the use of chimeric recombinant antigens such as fusions of mannose binding protein (MBP) with an antigen of interest, wherein the monoclonal antibody which is conjugated to colloidal gold is directed to MBP, leaving the entire antigen of interest free to react with patient antibody to give a visible signal in a diagnostic test as shown schematically in FIG. 6.

### EXAMPLE 7

### Monomeric Antigen with Ligand Binding Site as Bridge

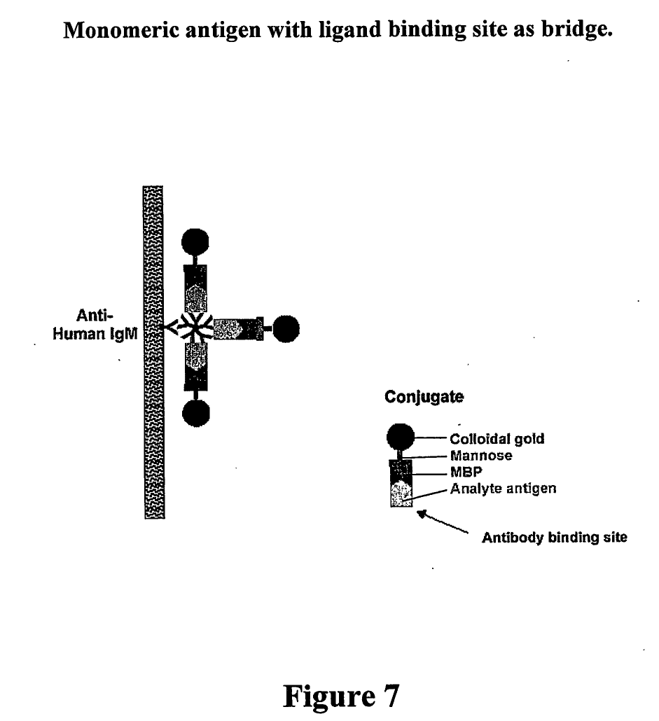

In this example the colloidal gold is chemically conjugated with mannose, to which MBP will bind because of its natural affinity for this ligand, leaving the immunodominant epitope(s) to react with patient antibody to give a visible signal in a diagnostic test as represented schematically in FIG. 7.

### EXAMPLE 8

### Multimeric Antigen of Hepatitis A Virus

### Detection Marker Connected X₂ Using a Protein:Protein Binding Molecule (Biotin:Anti-Biotin Antibody)

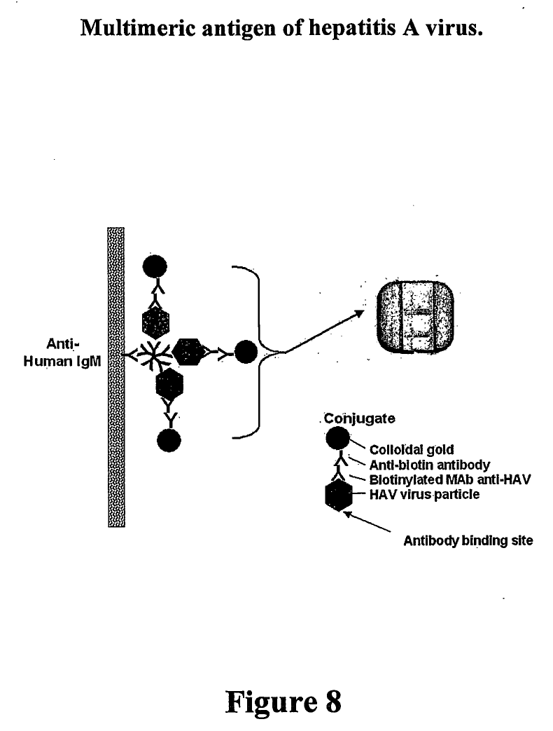

The colloidal gold-antibody conjugate is complexed with hepatitis A virus particles during performance of the assay, by bringing together the separate assay compartments containing the two parts. In this example, the colloidal gold-antibody conjugate may be formed by the use of colloidal gold conjugated to anti-biotin antibodies or streptavidin forming a complex with monoclonal antibody (K34C8 (MacGregor et al (supra))) conjugated to biotin via methods well known in the art. In this example, the monoclonal antibody (K34C8) may also be directed against the immunodominant epitope in the antigen, but under defined conditions such as virus concentration and/or time incubation only one or a few copies of the epitope within each virus particle will react with monoclonal antibody bound to the colloidal gold, leaving the remaining epitopes within the virus particle to react with patient antibody to give a visible signal in a diagnostic test as shown schematically in FIG. 8.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Anderson D. A. et al., J. Virol. Methods. 81:131-142, 1999;
Ausubel et al “Current Protocols in molecular Biology” John Wiley & Sons Inc, 1994-1998;
Bruss et al., J. Virol. 65:3813-3820, 1994;
Delpeyroux et al J. Mol. Biol. 195:343-350, 1987;
Eriksson et al. Biophys. J. 2: 64, 1993;
Hagensee et al., J. Virol. 67: 315, 1993;
Harlow and Lane, “Antibodies: A Laboratory Manual” Cold Spring Harbor Laboratory, 1988;
Kohler and Milstein, European Journal of Immunology 6: 511-519, 1976;
Lakowicz et al. Biophys. J 72: 567, 1997;
Li, F et al. J Med. Virol. 52:289-300, 1997;
Li, F. et al., J Med. Virol. 60:379-386, 2000;
MacGregor A. et al, J. Clin. Microbiol., 18(5):1237-1243, 1983;
Paul, Fundamental Immunology, 3rd ed., 243-247 (Raven Press, 1993);
Prange, et al, J. Gen. Virol. 76:2131-2140, 1995;
Riddell, M. A., et al J. Virol. 74:8011-8017, 2000;
Rose et al., J. Virol. 67: 1936, 1993;
Sambrook and Russell “Molecular Cloning—A Laboratory Manual” Cold spring Harbour Press, 2001;
Wild D., “The Immunoassay Handbook” Nature Publishing Group, 2001;
Youvan et al. (Biotechnology et elia 3: 1-18, 1997).

1. A method for detecting an antibody in a sample, the method comprising contacting the sample with components of a detection marker-antibody complex for a time and under conditions such that the detection marker is connected indirectly to an antigen recognised by the antibody to be detected by a bridging complex which preserves or enhances the availability of binding sites on the antigen for the antibody, and wherein the bridging complex comprises proteins X₁ and X₂ and wherein X₁ comprises a viral particle or viral-like particle, a dimeric or multimeric protein, or a chimeric or fusion protein which comprises an epitope recognised by the antibody to be detected and also binds reversibly to X₂, wherein X₂ is bound by X₁ and is also bound, fused or otherwise connected to the detection marker, and detecting the detection marker to indicate the presence of the antibody in the sample.

2. The method of claim 1, wherein components of the detection marker-antigen complex are stored or used separately or together.

3. The method of claim 2, wherein the detection marker-X₂ component and the X₁-antigen component are stored separately.

4. The method of claim 1, wherein the antibody is one or more of an IgM, IgE, IgA and IgG antibody.

5. The method of claim 1, wherein X₂ comprises an antibody, protein binding molecule, nucleic acid binding molecule, carbohydrate binding molecule or lipid binding molecule.

6. The method of claim 1, wherein X₁ is a viral particle comprising multiple copies of a binding site recognised by the antibody to be detected and wherein X₂ comprises an antibody which binds to the binding site, and wherein X₂ is contacted with X₁ such that only one or a few of the binding sites are bound leaving further binding sites on the virus particle to react with the antibody to be detected.

7. The method of claim 6, wherein X₁ is an isolated or recombinant hepatitis viral particle.

8. The method of claim 7, wherein the hepatitis viral particle is a hepatitis A viral particle.

9. The method of claim 7, wherein the hepatitis viral particle is a hepatitis B viral particle.

10. The method of claim 7, wherein the hepatitis viral particle is a hepatitis C viral particle.

11. The method of claim 7, wherein the hepatitis viral particle is a hepatitis E viral particle.

12. The method of claim 6, wherein X₁ and X₂ are stored separately and form a complex during performance of the method.

13. The method of claim 6, wherein the detection marker comprises one or more of a mass tag, dye, colloidal or magnetic-particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins, quantum dots, radioisotopes, redox complexes, substrates, viruses or other equivalent molecule.

14. The method of claim 1, wherein X₁ is an avian hepadnavirus virus-like particle (VLP).

15. The method of claim 14, wherein X₁ comprises multiple copies of a binding site recognised by the antibody to be detected and wherein X₂ comprises an antibody which binds to the binding site, and wherein X₂ is contacted with X₁ such that only one or a few of the binding sites are bound leaving further binding sites on the VLP to react with the antibody to be detected.

16. The method of claim 14, wherein X₂ binds to a binding site on the VLP not recognised by the antibody to be detected.

17. The method of claim 14, wherein the VLP is a recombinant duck hepadnavirus-like particle and X₂ is a monoclonal antibody determined by the S or L antigen of duck Hepadnavirus.

18. The method of claim 14, wherein the detection marker comprises one or more of a mass tag, dye, colloidal or magnetic-particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins quantum dots, radioisotopes, redox complexes, substrates, viruses or other equivalent molecule.

19. The method of claim 1, wherein X₁ is a dimeric or multimeric protein comprising at least two binding sites recognised by the antibody to be detected and wherein X₂ comprises an antibody which binds to the binding site, and wherein X₂ is contacted with X₁ such that only one or a few of the binding sites are bound leaving further binding sites on the dimer or multimer to react with the antibody to be detected.

20. The method of claim 19, wherein X₁ is dimeric ORF2.1 antigen of hepatitis E virus.

21. The method of claim 19, wherein the detection marker comprises one or more of a mass tag, dye, colloidal or magnetic-particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins, quantum dots, radioisotopes, redox complexes, substrates, viruses or other equivalent molecule.

22. The method of claim 1, wherein X₁ is a fusion or chimeric protein comprising the antigen and a second binding partner which binds reversibly to X₂, wherein X₂ comprises an antibody or a protein binding molecule or carbohydrate binding molecule or lipid binding molecule or nucleic acid binding molecule bound by the second binding partner.

23. The method of claim 22, wherein the second binding partner is a carbohydrate and X₂ comprises a carbohydrate binding protein.

24. The method of claim 22, wherein the second binding partner is a protein and X₂ comprises a protein binding protein.

25. The method of claim 22, wherein the detection marker is a mass tag, dye, colloidal particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins, quantum dots, radioisotopes, redox complexes, substrates, viruses or other equivalent molecule.

26. The method of claim 1, wherein the detection marker is a colloidal particle, such as colloidal gold, silver or selenium.

27. The method of claim 1, wherein the antibody is immobilised to a solid support prior to detection.

28. The method of claim 1 when used for detecting one or a plurality of specific antibodies in a sample.

29. The method of claim 1 when used for detecting one or a plurality of specific antibodies to hepatitis such as hepatitis A and/or B and/or C and/or E in a sample.

30. The method of claim 1, wherein the method is a chromatographic or immunochromatographic method.

31. A kit for detecting a specific antibody in a sample, in compartmental form comprising a portion to receive the sample and a portion to receive components of a detection marker-antigen complex, wherein the antigen comprises an epitope recognised by the antibody to be detected, if present in the sample, and wherein the detection marker is connected indirectly to the antigen by a bridging complex which preserves or enhances the availability of epitopes on the antigen for the antibody and detection thereof relative to a control, and wherein the bridging complex comprises bridge binding partners X₁ and X₂ wherein X₁ comprises a viral particle or virus-like particle, a dimeric or multimeric protein, or a chimeric or fusion protein which comprises an epitope recognised by the antibody to be detected and binds reversibly to X₂, wherein X₂ comprises an antibody or an protein binding molecule which is bound by X₁ and which is bound, fused or otherwise connected to the detection marker.

32. The kit of claim 31, wherein components of the detection marker-antigen complex are stored or used separately or together.

33. The kit of claim 31, wherein the detection marker-X₂ component and the X₁-antigen component are stored separately.

34. The kit of claim 31, wherein the specific antibody in the sample is one or more of an IgM, IgE, IgA and IgG antibody.

35. The kit of claim 31, wherein X₂ comprises an antibody, protein binding molecule, nucleic acid binding molecule, carbohydrate binding molecule or lipid binding molecule.

36. The kit of claim 31, wherein X₁ is a viral particle comprising multiple copies of a binding site recognised by the antibody to be detected and wherein X₂ comprises an antibody which binds to the binding site, and wherein X₂ is contacted with X₁ such that only one or a few of the binding sites are bound leaving further binding sites on the virus particle to react with the antibody to be detected.

37. The kit of claim 36, wherein X₁ is an isolated or recombinant hepatitis viral particle.

38. The kit of claim 37, wherein the hepatitis viral particle is a hepatitis A viral particle.

39. The kit of claim 37, wherein the hepatitis viral particle is a hepatitis B viral particle.

40. The kit of claim 37, wherein the hepatitis viral particle is a hepatitis C viral particle.

41. The kit of claim 37, wherein the hepatitis viral particle is a hepatitis E viral particle.

42. The kit of claim 36, wherein X₁ and X₂ are stored separately and form a complex during use of the kit.

43. The kit of claim 36, wherein the detection marker comprises one or more of a mass tag, dye, colloidal or magnetic-particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins, quantum dots, radioisotopes, redox complexes, substrates, viruses or other equivalent molecule.

44. The kit of claim 31, wherein X₁ is an avian hepadnavirus virus-like particle (VLP).

45. The kit of claim 44, wherein X₁ comprises multiple copies of a binding site recognised by the antibody to be detected and wherein X₂ comprises an antibody which binds to the binding site, and wherein X₂ is contacted with X₁ such that only one or a few of the binding sites are bound leaving further binding sites on the VLP to react with the antibody to be detected.

46. The kit of claim 44, wherein X₂ binds to a binding site on the VLP not recognised by the antibody to be detected.

47. The kit of claim 44, wherein the VLP is a recombinant duck hepadnavirus-like particle and X₂ is a monoclonal antibody determined by the S or L antigen of duck Hepadnavirus.

48. The kit of claim 44, wherein the detection marker comprises one or more of a mass tag, dye, colloidal or magnetic-particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins, quantum dots, radioisotopes, redox complexes, substrates, viruses or other equivalent molecule.

49. The kit of claim 31, wherein X₁ is a dimeric or multimeric protein comprising at least two binding sites recognised by the antibody to be detected and wherein X₂ comprises an antibody which binds to the binding site, and wherein X₂ is contacted with X₁ such that only one or a few of the binding sites are bound leaving further binding sites on the dimer or multimer to react with the antibody to be detected.

50. The kit of claim 49, wherein X₁ is dimeric ORF2.1 antigen of hepatitis E virus.

51. The kit of claim 49, wherein the detection marker comprises one or more of a mass tag, dye, colloidal or magnetic-particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins, quantum dots, radioisotopes, redox complexes, substrates, viruses or other equivalent molecule.

52. The kit of claim 31, wherein X₁ is a fusion or chimeric protein comprising the antigen and a second binding partner which binds reversibly to X₂, wherein X₂ comprises an antibody or a protein binding molecule or carbohydrate binding molecule or lipid binding molecule or nucleic acid binding molecule bound by the second binding partner.

53. The kit of claim 52, wherein the second binding partner is a carbohydrate and X₂ comprises a carbohydrate binding protein.

54. The kit of claim 52, wherein the second binding partner is a protein and X₂ comprises a protein binding protein.

55. The kit of claim 52, wherein the detection marker is a mass tag, dye, colloidal particle, enzyme, radioactive molecule, chemiluminophore, fluorophore, phosphorescent molecule, luminescent molecules such as firefly luciferase, metal and metalloid, metal complexes, microparticles, nucleic acids, phosphors, dielectric, paramagnetic and/or phosphorescent particles, photoproteins, quantum dots, radioisotopes, redox complexes, substrates, viruses or other equivalent molecule.

56. The kit of claim 31, wherein the detection marker is a colloidal particle, such as colloidal gold, silver or selenium.

57. The kit of claim 31, wherein the antibody is immobilised to a solid support prior to detection.

58. The kit of claim 31 when used for detecting one or a plurality of specific antibodies in a sample.

59. The kit of claim 31 when used for detecting one or a plurality of specific antibodies to hepatitis such as hepatitis A and/or B and/or C and/or E in a sample.

60. The kit of claim 31, wherein the kit is a chromatographic or immunochromatographic kit.
